(19) United States
(12) Patent Application Publication
(10) Pub. No.: US 2016/0303021 A1
(43) Pub. Date: Oct. 20, 2016

(54) COMPOSITION COMPRISING COLLAGEN PEPTIDE, ELASTIN PEPTIDE AND PROTEOGLYCAN

(71) Applicant: SUNTORY HOLDINGS LIMITED, Osaka-shi (JP)

(72) Inventors: Sayuri KITAGAWA, Osaka (JP); Nozomi KITAHARA, Osaka (JP); Taeko IINO, Osaka (JP)

(21) Appl. No.: 15/100,785

(22) PCT Filed: Nov. 28, 2014
(86) PCT No.: PCT/JP2014/081505

§ 371 Date: Jun. 1, 2016

(30) Foreign Application Priority Data
Dec. 4, 2013 (JP) 2013-250748

(51) Int. Cl. A61K 8/65; A23L 2/39; A23L 2/66; A61K 8/64; A61Q 19/08

(52) U.S. Cl. CPC A61K 8/65; A61K 8/64; A61Q 19/08; A23L 33/17; A23L 2/66; A23L 2/39; A61K 2800/5922; A23V 2002/00

(57) ABSTRACT

The present invention addresses the problem of providing a composition with which a skin beautifying effect can be demonstrated even if collagen peptide intake is reduced.

A composition that effectively demonstrates a skin beautifying effect, even with low-concentration collagen peptide, can be provided by making the weight ratio of collagen peptide, elastin peptide, and proteoglycan contained in the composition a prescribed ratio.

### TECHNICAL FIELD

The present invention relates to compositions comprising collagen peptide, elastin peptide and proteoglycan. More specifically, the present invention relates to compositions having a skin beauty improving effect that are characterized in that the weight of collagen peptide and the sum weight of elastin peptide and proteoglycan are at specified proportions.

### BACKGROUND ART

The skin is composed, from the outer surface inward, of the stratum corneum, the epidermis, the basement membrane and the dermis. The dermis which occupies the widest area in the skin is not so densely packed with cells as the epidermis. Rather it has a wide extracellular space and is filled with a network of macromolecules which is called an “extracellular matrix.” This extracellular matrix is directly involved in the elasticity, firmness, freshness, metabolism, etc. of the skin and it is known that symptoms of skin aging such as wrinkles and sag occur to connective tissue fibers in the dermis that are primarily composed of fibroblasts and the extracellular matrix. Since fibrous proteins such as collagen and elastin that compose the extracellular matrix and polysaccharides called acidic mucopolysaccharides such as hyaluronic acid and dermatan sulfate are produced by fibroblasts, promoting the proliferation of fibroblasts contributes to imparting firmness and gloss to the skin while preventing or ameliorating wrinkles and sag. From this viewpoint, a variety of beverages, cosmetics and the like have been proposed that contain components featuring an action for promoting the proliferation of fibroblasts.

Recently, collagen has been reported to have various physiological actions such as a bone reinforcing action that leads to prevention or amelioration of osteoporosis (Patent Document 1), an action for promoting the metabolism of a living tissue by reversing its lowered function due to aging (Patent Document 2), a skin metabolism promoting action and a skin activating action (Patent Document 3), and an action for preventing the aging of the skin with a view to preventing or ameliorating wrinkles (Patent Document 4); having these actions, collagen is widely used not only as a starting material for the production of cosmetics, foods and beverages but also as a functional biomaterial for use in pharmaceuticals.

However, in order for those physiological actions to be fully developed within the living body, collagen peptides need be ingested in large amounts and it has been reported that an effective daily dose that needs be ingested orally to develop a dry skin ameliorating action is 5-10 g for fish scale collagen peptide or 10 g or more for pig skin collagen peptide (Non-Patent Document 1). The decomposition product of collagen is known to be broken down to amino acids, dipeptides or tripeptides as it is digested and absorbed, and among them the dipeptides or tripeptides are shown to be effective. Further, it has been reported that collagen-derived hydroxyproline-containing dipeptides and tripeptides, when acted on skin fibroblasts, activate their proliferation to promote the production of collagen and hyaluronic acid (Non-Patent Document 2 and Patent Document 5).

### CITATION LIST

### Patent Literature

Patent Document 1: JPH 9-255588 A

Patent Document 2: JPH 7-278012 A

Patent Document 3: JPH 9-67262 A

Patent Document 4: JP 2005-314265 A

Patent Document 5: Japanese Patent No. 4995155

### Non-Patent Literature

Non-Patent Document 1: Nippon Shokuhin Kagaku Kogaku Kaishi (Journal of the Japanese Society for Food Science and Technology), March 2009, Vol. 56, No. 3, p. 137-145

Non-Patent Document 2: Journal of Dermatological Science, July 2007, Vol. 47, p. 102, 179

### SUMMARY OF INVENTION

### Technical Problem

As described above, useful effects can generally be exhibited when collagen is ingested in large amounts but in order to suppress the unwanted characteristic eating qualities of collagen, further technological development is desired that enables a more useful skin beauty improving effect to be exhibited by ingesting a smaller amount of collagen.

An object, therefore, of the present invention is to provide compositions that are capable of exhibiting a useful skin beauty improving effect even when the ingestion of collagen peptides is reduced.

### Solution to Problem

The present inventors conducted an intensive study with a view to solving the above-mentioned problem and found as a result that by ingesting compositions comprising collagen peptide, elastin peptide and proteoglycan at specified proportions, a skin beauty improving effect was efficaciously displayed even when the concentration of the collagen peptide was low. This finding has led to the accomplishment of the present invention.

The present invention encompasses but is not limited to the following embodiments.
(1) A composition comprising collagen peptide, elastin peptide and proteoglycan, wherein the weight ratio between the weight of collagen peptide and the sum weight of elastin peptide and proteoglycan [(collagen peptide):(elastin peptide+proteoglycan)] is between 1:10 and 1:0.0000001.
(2) The composition as recited in (1), wherein the weight ratio between the weight of collagen peptide and the weight of elastin peptide [(collagen peptide):(elastin peptide)] is between 1:5 and 1:0.000001.
(3) The composition as recited in (1), wherein the weight ratio between the weight of collagen peptide and the weight of proteoglycan [(collagen peptide):(proteoglycan)] is between 1:0.2 and 1:0.0000001.
(4) The composition as recited in any one of (1) to (3), which is for oral use.
(5) The composition as recited in any one of (1) to (3), which is a food or a beverage.
(6) A skin improving agent comprising collagen peptide, elastin peptide and proteoglycan, wherein the weight ratio between the weight of collagen peptide and the sum weight of elastin peptide and proteoglycan [(collagen peptide):(elastin peptide+proteoglycan)] is between 1:10 and 1:0.0000001.
(7) The skin improving agent as recited in (6), which has a fibroblasts proliferation promoting action.
(8) The skin improving agent as recited in (6) or (7), which has a collagen production promoting action.
(9) The skin improving agent as recited in any one of (6) to (8), which has an anti-skin aging action.

### Advantageous Effects of Invention

According to the present invention, there are provided compositions that efficaciously exhibit a skin beauty improving effect even if they have low concentrations of collagen peptide.

The compositions of the present invention are also advantageous in that they enable suppressing the amount of collagen peptide which presents an unwanted eating quality.

### DESCRIPTION OF EMBODIMENTS

Hereinafter, embodiments of the present invention are described in detail.

Composition

### <Active Ingredients>

First, the composition which is a mode of the present invention is characterized by comprising collagen peptide, elastin peptide and proteoglycan as active ingredients. The composition of the present invention brings about a skin beauty improving effect by having an action for promoting the proliferation of fibroblasts present in a living tissue, in particular, the dermis tissue of the skin.

(Collagen Peptide)

The collagen peptide to be used in the present invention may be obtained by subjecting collagen or modified collagen such as gelatin to hydrolysis treatments as by enzyme, acid, alkali, etc.; g; one or more of these materials may be employed. Examples of collagen and gelatin include ones derived from animals such as cattle, pig and chicken or derived from fish; in particular, collagen proteins that are extracted from the connective tissues in the skin, bone, tendon, etc. of animals, as well as from fish skin and scale may be employed.

Enzymes to be used to prepare collagen peptides may be of any types that are capable of cutting peptide bonds in collagen or gelatin and examples include collagenase, papain, bromelain, actinidin, ficin, cathepsin, pepsin, chymosin, trypsin, and enzyme preparations consisting of these enzymes in admixture. Examples of acids that may be used include hydrochloric acid, sulfuric acid, nitric acid, etc. Examples of alkalis that may be used include sodium hydroxide, calcium hydroxide, etc.

In the present invention, hydrolyzed collagen peptides may be used as they are in the form of an aqueous solution or they may be dried or otherwise reduced to a powder form. Alternatively, the aqueous solution may be subjected to a commonly employed treatment for purification and then used either as such or other forms including powder. Whichever of these forms is employed, the effects of the present invention will in no way be compromised.

While all molecular weights of collagen peptides can be used, typical are those which have molecular weights of no more than 10,000, for example, in the range of 100 to 7,000, preferably in the range of 100 to 5,000. The molecular weight of collagen peptides can be measured by known quantification methods such as HPLC and gel filtration. It should be noted here that the in vivo absorbability of collagen peptides generally decreases with the increasing molecular weight and vice versa but collagen peptides with smaller molecular weights present unwanted characteristic eating qualities (e.g. bitterness and harshness). In the present invention, the use of collagen peptides can be suppressed to lower levels by combining them with elastin peptide and proteoglycan and, hence, even if collagen peptides of relatively small molecular weights which have good in vivo absorbability are used, the problem of the unwanted characteristic eating qualities is unlikely to occur.

Collagen peptides may be used either as an extract or in purified form and it is preferred to use products with a purity of at least 50 wt %, more preferably with a purity of at least 70 wt %, and even more preferably with a purity of at least 90 wt %. Commercial products of collagen peptide may also be used, as exemplified by COLLAGEN PEPTIDE SCP-5100 (product of Nitta Gelatin Inc.)

The content of collagen peptides in the composition of the present invention is difficult to specify uniquely since it varies with the type of the starting materials to be combined with it, their contents and other factors but it may range from 1 wt % to 99.9 wt %, preferably from 30 wt % to 99.9 wt %, and more preferably from 50 wt % to 90 wt %. The intended effects of the present invention are not attainable if the content of collagen peptides is below 1 wt % or greater than 99.9 wt %.

(Elastin Peptide)

The elastin peptide as referred to in the present invention means water-soluble elastin peptide. Elastin peptides that can be used include extracts from the living tissues of animals such as cattle, pig, chicken, sheep and fish, as well as water-soluble or insoluble elastin that is hydrolyzed or otherwise treated for degradation with enzymes, acids, alkalis, etc. and, alternatively, artificially synthesized products may be used; and one or more of these materials may be employed.

The molecular weight of the elastin peptides to be used in the present invention is not particularly limited and all molecular weights of elastin may be used.

Elastin peptides may be used either as an extract or in purified form and it is preferred to use products with a purity of at least 50 wt %, more preferably with a purity of at least 70 wt %, and even more preferably with a purity of at least 90 wt %. Commercial products of elastin peptide may also be used, as exemplified by BIDAN ELASTIN FI (product of Nippon Suisan Kaisha, Ltd.), TUNA ELASTIN HS-1 (product of Hagoromo Foods Corporation), and BONITO ELASTIN (product of Hayashikane Sangyo Co., Ltd.)

The content of elastin peptides in the composition of the present invention varies with the type of the starting materials such as collagen peptide to be combined with it, their contents and other factors but it may range from 0.0000002 wt % to 10 wt %, preferably from 0.000002 wt % to 1 wt %, and more preferably from 0.000004 wt % to 0.2 wt %.

(Proteoglycan)

The proteoglycan to be used in the present invention is not particularly limited and examples that can be used are extracts from the cartilage of animals such as shark, whale, salmon and ray.

The molecular weight of the proteoglycan to be used in the present invention is not particularly limited but it may typically range from 10,000 to 10,000,000, preferably from 100,000 to 1,000,000.

Proteoglycan may be used either as an extract or in a purified form and it is preferred to use products with a purity of at least 5 wt %, more preferably with a purity of at least 10 wt %, and even more preferably with a purity of at least 15 wt %. Commercial products of proteoglycan may also be used.

The content of proteoglycan in the composition of the present invention varies with the type of the starting materials such as collagen peptide to be combined with it, their contents and other factors but it may range from 0.0000001 wt % to 5 wt %, preferably from 0.000001 wt % to 0.5 wt %, and more preferably from 0.000002 wt % to 0.1 wt %.

(Weight Ratio of Active Ingredients)

In the composition of the present invention, the weight ratio between the weight of collagen peptide and the sum weight of elastin peptide and proteoglycan [(collagen peptide):(elastin peptide+proteoglycan)] is typically between 1:10 and 1:0.0000001, preferably between 1:5 and 1:0.000001, and more preferably between 1:2 and 1:0.00001. The present inventors obtained the finding that a formulation comprising collagen peptide as combined with elastin peptide and proteoglycan had a higher skin improving effect than a formulation solely comprising the same dosage of collagen peptide. Therefore, it is anticipated that ingesting compositions comprising the active ingredients at weight ratios in the above-noted ranges will provide higher skin improving effects than when the same dosage of collagen peptide is ingested. Since lower concentrations of collagen peptide are sufficient to show an efficacious skin improving effect, the unwanted characteristic eating qualities of collagen peptide can be suppressed by ingesting compositions comprising the active ingredients at weight ratios in the above-noted ranges.

In the composition of the present invention, the weight ratio between the weight of collagen peptide and the weight of elastin peptide [(collagen peptide):(elastin peptide)] is typically between 1:5 and 1:0.000001, preferably between 1:2 and 1:0.00001, and more preferably between 1:1 and 1:0.0001; in the composition, the weight ratio between the weight of collagen peptide and the weight of proteoglycan [(collagen peptide):(proteoglycan)] is typically between 1:0.2 and 1:0.0000001, preferably between 1:0.1 and 1:0.0000005, and more preferably between 1:0.02 and 1:0.000002. The present inventors also obtained an unexpected finding by experiment that a particularly high collagen production promoting effect was exhibited when collagen peptide was combined with elastin peptide and proteoglycan at low proportions. To be more specific and assuming that the same dosage of collagen peptide is ingested, a higher skin improving effect is obtained by a composition having lower weight ratios of elastin peptide and proteoglycan. Therefore, according to the present invention, the amounts of the respective active ingredients can be reduced to very low levels, enabling the production of compositions that are easy to ingest perorally and which feature high cost effectiveness.

### <Other Ingredients>

Aside from the active ingredients described above, known additives may be incorporated in the compositions of the present invention. Although the applicable additives are not particularly limited, those which are commonly used for oral ingestion are preferred and examples that can be used include excipients, binders, disintegrants, lubricants, antiseptics, flavoring agents, aroma corrigents, coloring agents, scents, etc. and any ingredients can be used that are known to have skin improving effects.

In the compositions of the present invention, other beautifying or health promoting components that are known to have skin improving effects can be incorporated in combination with the active ingredients. The components that can be used in combination with the active ingredients are not particularly limited and examples include ceramides, lactobacilli, vitamins (e.g. vitamin C), minerals (e.g. calcium), plant extracts, etc.

Further in addition, components that are exemplified below which are known to have a fibroblast proliferation promoting action, a collagen production promoting action, and an anti-skin aging action may be incorporated in the compositions of the present invention.

### <Form and Dosage>

The composition of the present invention is preferably an oral composition. The oral composition embraces, but is not limited to, for example, foods and beverages, pharmaceuticals and quasi-drugs, etc.; the oral composition is preferably a food and a beverage, more preferably a beverage. Exemplary foods and beverages include health foods, therapeutic foods, foods for specified health use, dietary supplements, dairy products, soft drinks, powders ready for dissolving just before use, etc; foods and beverages for pets, feeds for livestock, etc. are also included. Particularly preferred are powders ready for dissolving just before use. If desired, the powders ready for dissolving just before use may be dissolved in coffee, tea, juice, yoghurt, soup, etc. or mixed in cooked food and then ingested. The powders ready for dissolving just before use are particularly superior in terms of stability during storage and the ease of administration, i.e., they can be ingested after being dissolved in or added to beverages or foods of preference. If desired, the composition of the present invention can be used in the form of tablets or capsules. Pharmaceuticals and quasi-drugs are typically in the form of oral preparations such as granules, tablets, capsules, and liquids/solutions.

The dosage of the composition of the present invention can be determined as appropriate depending on the subject's age, body weight, health conditions, etc; the daily intake by a human adult of the composition typically ranges from 10 mg to 100,000 mg, preferably from 500 mg to 15,000 mg, and more preferably from 3,000 mg to 10,000 mg, and the composition can be ingested or administered either in a single dose or in divided portions. As the collagen peptide need be used in a lower dosage, it has become possible to reduce the required daily intake of the composition.

### <Actions and Effects>

The compositions of the present invention can be used to prevent or ameliorate symptoms of the skin and they are capable of preventing or ameliorating symptoms of the skin such as, for example, lowered skin moisture retention and elasticity, reduced skin firmness and gloss, rough skin, wrinkles, and dullness.

Here it is known that the skin improving effects are exhibited by the action for promoting the proliferation of fibroblasts found in living tissues, in particular, the dermis tissue of the skin, and for that matter, the associated collagen production promoting action and anti-skin aging action. Therefore, in another embodiment of the present invention, the composition has the fibroblasts proliferation promoting action, the collagen production promoting action, or the anti-skin aging action.

The fibroblasts proliferation promoting action means the action for promoting the proliferation of fibroblasts found in living tissues, in particular, the dermis tissue of the skin and it can be evaluated by known methods. The active ingredients capable of the fibroblasts proliferation promoting action are collagen peptide, elastin peptide and proteoglycan. These ingredients, after being processed into a solid, paste or liquid form, may immediately be applied as a skin improving agent that has the fibroblasts proliferation promoting action; if desired, the agent may be combined with known additives having the fibroblasts proliferation promoting action which are included in the agent in the usual manner to formulate a composition. Ingredients that are known to have the fibroblasts proliferation promoting action may include, but are not limited to: those which are disclosed in the documents listed above as prior art references; as well as dried products or extracts of plants and algae such as chlorella, aloe barbadensis, rice, jujube, Alpinia zerumbet, Curcuma amada, Ampelopsis brevipedunculata, Adiantum capillus-veneris, Nelumbo nucifera germ, sesame, pepper, Angelica acutiloba, Houttuynia cordata, Lonicera caerulea var. emphyllocalyx fruit, Mallotus philippensis (Lam.) Muell. Arg., algae (Caulerpa racemosa), Rubus ellipticus, and Coix lacryma-jobi var. ma-yuen; catechins, imino-group containing peptides, α-lipoic acid and its derivatives such as salts, esters, amides, etc., dihydrolipoic acid and its derivatives, chitin hydrolyzates, N-acetyl-D-glucosamine and its oligomers.

The collagen production promoting action refers to the action for potentiating the production of collagen in vivo, and the production of collagen is more desirably that of collagen by fibroblasts in the dermis tissue. The production of collagen can be evaluated by, for example, measuring the production level of type I collagen by known methods; alternatively, the production of collagen may be confirmed by evaluating the expression level of a collagen synthesis gene (COL1a1 gene) in human skin fibroblasts by known methods.

The anti-skin aging action means preventing symptoms of the aging of the skin, which include at least one symptom selected from the group consisting of skin wrinkles, blotches, dullness, freckles, sagging, open pores, dry skin, and rough skin.

The present inventors have confirmed through experiment that by using collagen peptide, elastin peptide and proteoglycan in combination, not only can the proliferation of human skin fibroblasts be promoted but also the expression of the collagen synthesis gene can be enhanced as compared with the case of using collagen peptide alone; the inventors have also confirmed the resulting marked promotion of collagen production. From these findings, any skilled artisan should understand that even if the intake of collagen peptide is made smaller than when it is conventionally ingested, marked skin improving effects are obtained by ingesting collagen peptide, elastin peptide and proteoglycan in combination.

### <Production Method>

While the composition of the present invention can be produced in the usual manner, the active ingredients collagen peptide, elastin peptide and proteoglycan may be incorporated either by itself or after being processed into a solid, paste or liquid form. If desired, other components such as known ingredients and additives having the skin improving action may optionally be incorporated. The contents and weight ratios of the active ingredients and other components are as described above.

For example, collagen peptide, elastin peptide and proteoglycan may optionally be mixed with edible ingredients such as excipients (e.g. glucose, dextrin, lactose, starch or its processed products, and cellulose powder), vitamins, minerals, fats and oils from animals, plants, fishes and shells, proteins (e.g. animal-, plant- or yeast-derived proteins, and their hydrolyzates), saccharides, pigments, scents, antioxidants, surfactants, other food additives, a variety of ingredients with nutrient function claims, as well as ceramides, casein, etc. in powder or extract form, and the mixtures are either processed into powder, granular, pellet, tablet and other forms or processed into the above-mentioned foods and beverages in the usual manner; if desired, these mixtures in liquid form may be coated with gelatin, sodium alginate, carboxymethyl cellulose or other coating agents to mold capsules or, alternatively, they may be processed into the form of beverages (drinks). In particular, therapeutic foods and health foods are preferred products of processing, and powders ready for dissolving just before use are more preferred.

Skin Improving Agent

A further embodiment of the present invention is a skin improving agent comprising collagen peptide, elastin peptide and proteoglycan in specified proportions. In the skin improving agent, the weight ratio between the weight of collagen peptide and the sum weight of elastin peptide and proteoglycan [(collagen peptide):(elastin peptide+proteoglycan)] is between 1:10 and 1:0.0000001, preferably between 1:5 and 1:0.000001, and more preferably between 1:2 and 1:0.00001. In the skin improving agent, the weight ratio between the weight of collagen peptide and the weight of elastin peptide [(collagen peptide):(elastin peptide)] is typically between 1:5 and 1:0.000001, preferably between 1:2 and 1:0.00001, and more preferably between 1:1 and 1:0.0001. Further, in the skin improving agent, the weight ratio between the weight of collagen peptide and the weight of proteoglycan [(collagen peptide):(proteoglycan)] is typically between 1:0.2 and 1:0.0000001, preferably between 1:0.1 and 1:0.0000005, and more preferably between 1:0.02 and 1:0.000002. Regarding the active ingredients and other components to be contained in the skin improving agent, its form and dosage, actions and effects, as well as the method of its production, the information given above concerning the composition may apply.

The term “skin improving agent” as used herein means preparations for preventing or ameliorating symptoms of the skin such as lowered skin moisture retention and elasticity, reduced skin firmness and gloss, rough skin, wrinkles and dullness.

The ability of the skin improving agent to prevent or ameliorate the various symptoms of the skin may be associated with at least one action selected from the group consisting of the fibroblast proliferation promoting action, the collagen production promoting action, and the anti-skin aging action.

### EXAMPLES

On the following pages, the present invention will be described in greater detail based on Examples, to which the present invention is by no means limited.

### Example 1

Study on the Action for Promoting the Proliferation of Human Skin Fibroblasts

The effects to be exerted by the fibroblast proliferation promoting agent of the present invention on the proliferation of skin fibroblasts were examined by the following method.

### <Preparation of Test Samples>

Collagen peptide, elastin peptide and proteoglycan were weighed, dissolved in sterilized water, sterilized by passage through a membrane filter with a pore size of 0.2 μm, and diluted 100-fold with a 0.1% FBS containing DMEM medium to give the concentrations indicated in Table 1; the thus prepared samples were designated Samples 2 to 6. Used as a control was Sample 1 prepared by diluting sterilized water 100-fold with the 0.1% FBS containing DMEM medium. The indicated concentrations for the respective test samples were calculated as the concentrations of the individual ingredients. Details of the starting materials used are identified below.

Collagen peptide: product of Nitta Gelatin Inc. with a molecular weight of 100-1800 and a purity of 100%

Elastin peptide: BIDAN ELASTIN FI (product of Nippon Suisan Kaisha, Ltd.) with a purity of 100%

Proteoglycan: PROTEOGLYCAN F (product of ICHIMARU PHARCOS Co., Ltd.) with a molecular weight of ca. 450,000 and a purity of 20%

### <Method of Evaluation>

An adult-derived normal human dermal fibroblast NHDF strain (purchased from KURABO INDUSTRIES LTD.) was inoculated in a 10% fetal bovine serum (FBS) containing Dulbecco's Modified MEM medium (DMEM, product of NISSUI), seeded in a 96-well plate at a density of 10,000 cells/well, and cultured overnight under the conditions of 37° C. and 5% CO₂. The test samples, after being dissolved in water, were diluted 100-fold with the 0.1% FBS containing DMEM medium as described above and then added to the cells. Percent cell survival was measured 24 hours after the addition of each test sample (200 μL); it was calculated from the amount of formazan that formed as the result of reduction with an intracellular dehydrogenase in an MTS reagent (product of Promega). The percent cell survival for the respective test samples was determined as a value (%) relative to the case where the percent cell survival for the sample prepared by adding only the 0.1% FBS containing DMEM medium in the presence of 1% water was taken as 100%. The amount of formazan was measured by UV absorption at 490 nm. The present inventors conducted a preliminary study to determine the FBS concentration at which cells neither proliferated or decreased in number and upon confirming that this condition was satisfied by performing culture in the 0.1% FBS containing DMEM medium, they adjusted the serum concentration of the test medium to 0.1% to thereby design a method for quantitative evaluation of the fibroblast proliferation promoting action of test samples alone.

### <Results of Evaluation>

The results of evaluation are shown in Table 1. From the data in Table 1, the present inventors confirmed that collagen peptide as combined with elastin peptide in certain proportions was more effective than collagen peptide alone in promoting the proliferation of fibroblasts and that an even higher proliferation promoting effect was obtained when collagen peptide was combined with not only elastin peptide but also proteoglycan. From the results with Samples 5 and 6, it was revealed that fibroblasts were markedly increased in number even when the ratio of proteoglycan to collagen peptide was very low.

TABLE 1

| Added ingredients and their concentrations (μg/mL) | | | | Human dermal |
|---|---|---|---|---|
| | collagen | elastin | | fibroblast count |
| | peptide | peptide | proteoglycan | (relative value) |
| Sample 1 | 0 | 0 | 0 | 100.0 ± 3.18 |
| (control) | | | | |
| Sample 2 | 10 | 0 | 0 | 100.8 ± 2.87 |
| Sample 3 | 10 | 1 | 0 | 105.9 ± 2.22 |
| Sample 4 | 10 | 10 | 0 | 104.7 ± 1.33 |
| Sample 5 | 10 | 1 | 0.02 | 107.7 ± 1.81 |
| Sample 6 | 10 | 10 | 0.2 | 111.2 ± 1.27 |

### Example 2

Study on the Action for Promoting Collagen Production from Human Skin Fibroblasts

### <Preparation of Test Samples>

Samples 1 to 7 identified in Table 2 below were prepared in accordance with the method employed in Example 1.

### <Method of Evaluation>

An NHDF strain was inoculated in a 10% FBS containing DMEM medium, seeded in a 24-well plate at a density of 125,000 cells/well, and cultured overnight under the conditions of 37° C. and 5% CO₂. The test samples, after being dissolved in water, were diluted 100-fold with a 0.1% FBS containing DMEM medium and then added to the cells. Collagen production level was measured 72 hours after the addition of each test sample. To determine the collagen production level, type I collagen secreted into the supernatant of cell culture was quantified using Human Collagen Type I ELISA Kit (product of ACEL, Inc.) After color development using a polyclonal antibody specific for atelocollagen, measurement was conducted by UV absorption at 450 nm. The collagen production level for the respective test samples was determined as a value (%) relative to the case where collagen production level for the sample prepared by adding only the 0.1% FBS containing DMEM medium in the presence of 1% water was taken as 100%. During cell culture, the serum concentration in the culture broth was held low as in Example 1 to thereby reduce its possible effect on collagen synthesis.

### <Results of Evaluation>

The results of evaluation are shown in Table 2. From the data in Table 2, the present inventors confirmed that collagen peptide as combined with elastin peptide and proteoglycan was even more effective than collagen peptide alone in promoting the production of collagen. Since Sample 3 containing elastin peptide and proteoglycan at the lowest concentrations produced collagen in the greatest amount, the present inventors also found that the lower the concentrations of elastin peptide and proteoglycan, the stronger the action for promoting the production of collagen.

TABLE 2

| Added ingredients and their concentrations (μg/mL) | | | | Type I collagen |
|---|---|---|---|---|
| | collagen | elastin | | production level |
| | peptide | peptide | proteoglycan | (relative value) |
| Sample 1 | 0 | 0 | 0 | 100.0 |
| (control) | | | | |
| Sample 2 | 10 | 0 | 0 | 100.4 |
| Sample 3 | 10 | 0.001 | 0.00002 | 199.0 |
| Sample 4 | 10 | 0.01 | 0.0002 | 137.6 |
| Sample 5 | 10 | 0.1 | 0.002 | 147.2 |
| Sample 6 | 10 | 1 | 0.02 | 135.5 |
| Sample 7 | 10 | 10 | 0.2 | 105.5 |

### Example 3

Effects on the Expression of Various Genes in Human Skin Fibroblasts

### <Preparation of Test Samples>

Samples 1 to 4 identified in Table 3 below were prepared in accordance with the method employed in Example 1.

### <Method of Evaluation>

An NHDF strain was inoculated in a 10% FBS containing DMEM medium, seeded in a 96-well plate at a density of 10,000 cells/well, and cultured overnight under the conditions of 37° C. and 5% CO₂. The test samples, after being dissolved in water, were diluted 100-fold with a 0.1% FBS containing DMEM medium and then added to the cells. The expression of type I collagen synthesis gene (COL1a1) was measured by real-time quantitative PCR after the passage of 24 hours following the addition of each test sample. β-actin was used as a control housekeeping gene. Cells were washed with PBS and cDNA was constructed in accordance with the protocol for TaqMan Gene Expression Cell-to-CT Kit (Ambion). With this template cDNA, real-time PCR was performed using Taq Man Fast Univ. Gene Expression Mater Mix (Applied Biosystems). For real-time PCR, StepOnePlus Real Time PCR System (Applied Biosystems) was employed. Analysis was done by the ΔΔCT method and the results were expressed by ratios with unity (“1”) being written for the compensated value for the cells cultured in the absence of added any test sample.

### <Results of Evaluation>

The results of evaluation are shown in Table 3. From the data in Table 3, the present inventors confirmed that collagen peptide as combined with elastin peptide and proteoglycan was more effective than collagen peptide alone in enhancing the expression of the collagen synthesis gene.

TABLE 3

| Added ingredients and their concentrations (μg/mL) | | | | COL1a1 gene |
|---|---|---|---|---|
| | collagen | elastin | | expression |
| | peptide | peptide | proteoglycan | (relative value) |
| Sample 1 | 0 | 0 | 0 | 100 |
| (control) | | | | |
| Sample 2 | 10 | 0 | 0 | 118.5 |
| Sample 3 | 10 | 0.01 | 0.0002 | 143.7 |
| Sample 4 | 10 | 0.1 | 0.002 | 130.3 |

Production 1: Powder Ready for Dissolving Just Before Use

Collagen, elastin, proteoglycan and dextrin were weighed in the amounts indicated in Table 4 below and uniformly mixed to produce a powder ready for dissolving just before use (6.5 g). The powder thus obtained was dissolved in water, fruit juice or consommé soup; because of its good dispersibility in all media, the powder was suitable as a beverage ready for dissolving just before use.

TABLE 4
<Composition of the powder ready for dissolving just before use>

| Ingredient | Amount (mg) | Relative content (wt %) |
|---|---|---|
| Collagen peptide | 5000.0 | 76.92 |
| Elastin peptide | 1.0 | 0.02 |
| Proteoglycan | 0.5 | 0.01 |
| Dextrin | 1498.5 | 23.05 |
| Total | 6500.0 | 100.00% |

Production 2: Capsule

A soft capsule shell consisting of the components indicated below was filled in the usual manner with the above-described powder ready for dissolving just before use, whereby soft-shelled capsules were produced.

| | |
|---|---|
| Gelatin | 60.0% |
| Glycerin | 30.0% |
| Methyl paraoxybenzoate | 0.15% |
| Propyl paraoxybenzoate | 0.51% |
| Water | q.s. |

Production 3: Tablet

A mixture of the recipe identified below was granulated and molded in the usual manner to produce tablets.

TABLE 5

| Ingredient | Amount (mg) | Relative content (wt %) |
|---|---|---|
| Collagen peptide | 5000.0 | 50.00 |
| Elastin peptide | 0.1 | 0.001 |
| Proteoglycan | 0.05 | 0.0005 |
| Crystalline cellulose | 2950.0 | 29.5 |
| Partly pregelatinized starch | 2000.0 | 20.0 |
| Magnesium stearate | 49.85 | 0.499994 |
| Total | 10000.0 | 100.00% |

### INDUSTRIAL APPLICABILITY

By ingesting compositions comprising collagen peptide, elastin peptide and proteoglycan at specified proportions, a useful skin beauty improving effect can be displayed even when the concentration of the collagen peptide is low.

1. A composition comprising collagen peptide, elastin peptide and proteoglycan, wherein the weight ratio between the weight of collagen peptide and the sum weight of elastin peptide and proteoglycan [(collagen peptide):(elastin peptide+proteoglycan)] is between 1:10 and 1:0.0000001.

2. The composition according to claim 1, wherein the weight ratio between the weight of collagen peptide and the weight of elastin peptide [(collagen peptide):(elastin peptide)] is between 1:5 and 1:0.000001.

3. The composition according to claim 1, wherein the weight ratio between the weight of collagen peptide and the weight of proteoglycan [(collagen peptide):(proteoglycan)] is between 1:0.2 and 1:0.0000001.

4. The composition according to claim 1, which is for oral use.

5. The composition according to claim 1, which is a food or a beverage.

6. A skin improving agent comprising collagen peptide, elastin peptide and proteoglycan, wherein the weight ratio between the weight of collagen peptide and the sum weight of elastin peptide and proteoglycan [(collagen peptide):(elastin peptide+proteoglycan)] is between 1:10 and 1:0.0000001.

7. The skin improving agent according to claim 6, which has a fibroblasts proliferation promoting action.

8. The skin improving agent according to claim 6, which has a collagen production promoting action.

9. The skin improving agent according to claim 6, which has an anti-skin aging action.
